(19) United States
(12) Patent Application Publication
(10) Pub. No.: US 2026/0309666 A1
(43) Pub. Date: Oct. 8, 2026

(54) MICROCHIP GAS SENSING CAPACITOR

(71) Applicants: Serinus Labs, Inc., Berkeley, CA (US); The Regents of the University of California, Oakland, CA (US)

(72) Inventors: Hossain Fahad, Berkeley, CA (US); Thach Nguyen, Pinhole, CA (US); David W. Gardner, Richmond, CA (US); Gordon A. Charles, Sunnyvale, CA (US); Ali Javey, Lafayette, CA (US)

(73) Assignees: Serinus Labs, Inc., Berkeley, CA (US); The Regents of the University of California, Oakland, CA (US)

(21) Appl. No.: 19/170,324

(22) Filed: Apr. 4, 2025

(51) Int. Cl. G01N 27/22; G01N 33/00

(52) U.S. Cl. CPC G01N 27/226; G01N 27/227; G01N 33/0073

(57) ABSTRACT

A gas sensing microchip device includes a substrate layer and a gas capacitor (GASCAP) having a first electrically conductive plate configured to detect a gas. GASCAP also includes a gas-insensitive, electrically conductive element electrically connected to the first plate and having a first electrical terminal. GASCAP additionally includes a gas-insensitive second electrically conductive plate having a second electrical terminal and a high permittivity dielectric layer arranged between the first and second plates. The high permittivity dielectric layer induces a capacitive charge distribution on the second plate and an electrical bias between the first and second plates in response to the detected gas. The electrical bias generates a transient electrical current flow between the first and second terminals corresponding to the detected gas amount. The device also includes a low permittivity dielectric layer between the substrate layer and the second plate, isolating the GASCAP thermally and electrically from the substrate.

### GOVERNMENT LICENSE RIGHTS

This invention was made with United States Government support under SBIR Phase II program contract No.: 2112273 awarded by the National Science Foundation. The United States Government has certain rights in this invention.

### INTRODUCTION

The present disclosure generally relates to a miniaturized gas sensing microchip device operating as a gas capacitor.

A sensor is a device that produces an output signal for the purpose of sensing a physical phenomenon. In the broadest definition, a sensor is a device, module, machine, or subsystem that detects events or changes in its environment and sends the information to other electronics, frequently a computer processor. Sensors are usually designed to have a small effect on what is measured; making the sensor smaller often improves this characteristic and may introduce other advantages.

With advances in micromachinery and easy-to-use microcontroller platforms, the uses of sensors have expanded beyond the traditional fields of temperature, pressure, and flow measurement, for example into microchip-based integrated circuit sensors. In most cases, microchip-based sensors reach a significantly faster measurement time and higher sensitivity compared with macroscopic approaches. Microchip-based sensors may be effective in detecting concentrations of chemical substances, such as gases.

With the proliferation of various electrically powered systems and devices, a growing need has emerged for more effective monitoring of performance and state of health of energy storage arrays, such as battery packs and modules. Similar to other systems, over the course of their operating life, batteries emit gases. Highly sensitive microchip-based chemical sensors requiring ultra-low power and exhibiting low drift and long life may therefore provide advantageous means for monitoring gas emissions in energy storage applications, as well as in other systems.

### SUMMARY

A gas sensing microchip device having a substrate layer and a gas capacitor (GASCAP). The GASCAP includes a first electrically conductive plate configured to interact with and detect at least one gas via an exposed gas sensing surface. The GASCAP also includes a gas-insensitive, electrically conductive element electrically connected to the first electrically conductive plate (either electrically grounded or set to a fixed potential) and having a first electrical terminal. The GASCAP additionally includes a gas-insensitive second electrically conductive plate having a second electrical terminal. The GASCAP also includes a comparatively high permittivity dielectric layer arranged between the first and second electrically conductive plates.

The comparatively high permittivity dielectric layer is configured to induce a capacitive charge distribution on the gas-insensitive second electrically conductive plate and an electrical bias between the first and second electrically conductive plates in response to the gas(es) being detected by the first electrically conductive plate. The electrical bias in turn generates a transient electrical current flow between the first and second electrical terminals corresponding to an amount of the gas(es) being detected. The microchip device also includes a comparatively low permittivity dielectric layer arranged between the substrate layer and the second electrically conductive plate and configured to isolate the GASCAP thermally and electrically from the substrate layer.

The first electrically conductive plate may be constructed from a metal defined by a work-function property reversibly responsive to the gas(es). The subject metal may, for example, be Palladium, Platinum, Gold, Silver, Ruthenium, or Silver/Gold.

The first electrically conductive plate may extend to the comparatively low permittivity dielectric layer and thereby encapsulate, i.e., cover in its entirety, the comparatively high permittivity dielectric layer.

The first electrically conductive plate and the electrically conductive element together define a first capacitor plate area and the gas-insensitive second electrically conductive plate defines a second capacitor plate area. In such an embodiment, the first capacitor plate area may be greater than the second capacitor plate area, thereby enhancing sensitivity of the GASCAP to the gas(es).

The gas-insensitive second electrically conductive plate and the electrically conductive element may be constructed from the same metal.

The first electrically conductive plate may have an electrically discontinuous, e.g., nanoparticle or nanowire, structure.

The gas sensing microchip device may additionally have a gas-insensitive electrically conductive charge transfer layer arranged in direct contact with the electrically conductive element, between the electrically discontinuous first electrically conductive plate and the dielectric layer. In such an embodiment, the conductive charge transfer layer may be configured to electrically connect the discontinuous first electrically conductive plate to ground or may be set to a fixed potential.

Alternatively, the first electrically conductive plate may have an electrically continuous, e.g., flat sheet or mesh, structure.

The gas sensing surface of the first electrically conductive plate may be non-planar and thereby configured to enhance total capacitance of the GASCAP without increasing overall volume and packaging space occupied by the microchip device.

The gas sensing microchip device may additionally include a variable capacitance device operatively connected to the second electrical terminal. In such an embodiment, the variable capacitance device may be configured to respond with a change in threshold voltage indicative of the capacitive charge distribution on the gas-insensitive second electrically conductive plate.

The variable capacitance device may be arranged on or mounted to the substrate layer, thereby generating an integrated, monolithic system-on-chip (SoC).

The gas sensing microchip device may further include a reference capacitor arranged proximately to the GASCAP, in operative communication with the variable capacitance device, and configured to compensate for electronic drift of the GASCAP during detection of the gas(es).

The gas sensing microchip device may additionally include a microheater configured to generate thermal energy and thereby increase temperature of the GASCAP and the reference capacitor and a temperature sensor configured to detect temperature of the GASCAP.

The comparatively low permittivity dielectric layer may include a comparatively low permittivity dielectric layer base and a comparatively low permittivity dielectric layer island mechanically suspended thereon and separated therefrom by an airgap. The comparatively low permittivity dielectric layer island may be configured to support the GASCAP, the reference capacitor, the microheater, and the temperature sensor.

The gas sensing microchip device may further include a heat spreading structure embedded in the dielectric layer island and configured to generate a uniform temperature profile across the dielectric layer island.

The gas sensing microchip device may additionally include a guard ring arranged on the dielectric layer base surrounding each of the GASCAP and the reference capacitor. The guard ring may be configured to minimize stray charge and/or build-up or electromagnetic interference (EMI) on each of the GASCAP and the reference capacitor.

A gas sensing system having an electronic control unit (ECU) in operative communication with the gas sensing microchip device is also disclosed.

As part of the sensing system, the ECU is configured to ascertain the amount of the gas(es) being detected by the first electrically conductive plate and corresponding to the transient electrical current flow generated between the first and second electrical terminals and communicated to the ECU.

In the gas sensing system, the variable capacitance device may be arranged remotely from the gas sensing microchip device, such as on a separate microchip.

The above features and advantages, and other features and advantages of the present disclosure, will be readily apparent from the following detailed description of the embodiment(s) and best mode(s) for carrying out the described disclosure when taken in connection with the accompanying drawings and appended claims.

### BRIEF DESCRIPTION OF THE DRAWINGS

FIG. 1A is a schematic illustration of an embodiment of a sensing system including a plurality of gas sensing microchip devices in communication with an electronic control unit (ECU), according to the present disclosure.

FIG. 1B is a schematic illustration of another embodiment of the sensing system shown in FIG. 1A, according to the present disclosure.

FIG. 2 is a schematic cross-sectional side view of an embodiment of a gas sensing microchip device shown in FIGS. 1A and 1B, including a gas capacitor (GASCAP) incorporated into a microchip substrate and a variable capacitance device operatively connected to the GASCAP and integrated into the gas sensing microchip device, according to the present disclosure.

FIG. 3 is a schematic cross-sectional side view of another embodiment of the gas sensing microchip device shown in FIGS. 1A and 1B, including a reference capacitor, according to the present disclosure.

FIG. 4 is a schematic close-up cross-sectional side view of another embodiment of the gas sensing microchip device shown in FIGS. 1A and 1B, illustrating electrically discontinuous first electrically conductive plate of the GASCAP, according to the present disclosure.

FIG. 5 is a schematic cross-sectional side view of the of another embodiment of the gas sensing microchip device shown in FIGS. 1A and 1B, illustrating non-planar GASCAP construction, according to the present disclosure.

FIG. 6 is a schematic close-up cross-sectional side view of another embodiment of the gas sensing microchip device shown in FIGS. 1A and 1B, illustrating a comparatively low permittivity dielectric layer of the GASCAP including a comparatively low permittivity dielectric layer base and a mechanically suspended comparatively low permittivity dielectric layer island, according to the present disclosure.

FIG. 7 is a schematic top view of the GASCAP shown in FIG. 2, according to the present disclosure.

FIG. 8 is a plot of GASCAP transient current flow versus time, according to the present disclosure.

### DETAILED DESCRIPTION

Embodiments of the present disclosure as described herein are intended to serve as examples. Other embodiments may take various and alternative forms. Additionally, the drawings are generally schematic and not necessarily to scale. Some features may be exaggerated or minimized to show details of particular components. Therefore, specific structural and functional details disclosed herein are not to be interpreted as limiting, but merely as a representative basis for teaching one skilled in the art to variously employ the present disclosure.

Certain terminology may be used in the following description for the purpose of reference only and thus are not intended to be limiting. For example, terms such as “above” and “below” refer to directions in the drawings to which reference is made. Terms such as “front”, “back”, “fore”, “aft”, “left”, “right”, “rear”, “side”, “upward”, “downward”, “top”, and “bottom”, etc., describe the orientation and/or location of portions of the components or elements within a consistent but arbitrary frame of reference, which is made clear by reference to the text and the associated drawings describing the components or elements under discussion.

Furthermore, terms such as “first”, “second”, “third”, and so on may be used to describe separate components. Such terminology may include the words specifically mentioned above, derivatives thereof, and words of similar import, and are used descriptively for the figures, and do not represent limitations on the scope of the disclosure, as defined by the appended claims. Moreover, the teachings may be described herein in terms of functional and/or logical block components and/or various processing steps. It should be realized that such block components may include a number of hardware, software, and/or firmware components configured to perform the specified functions.

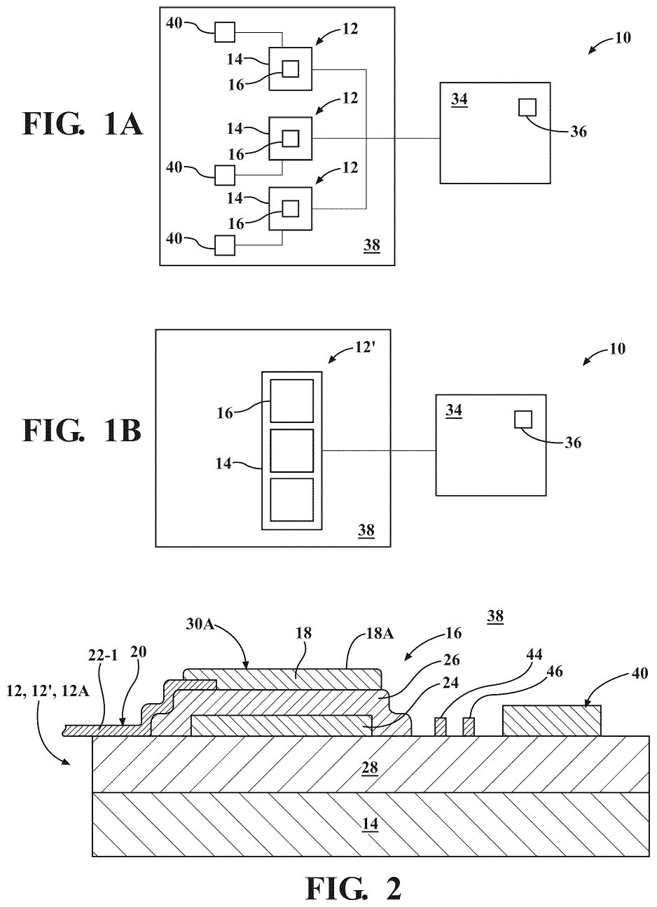

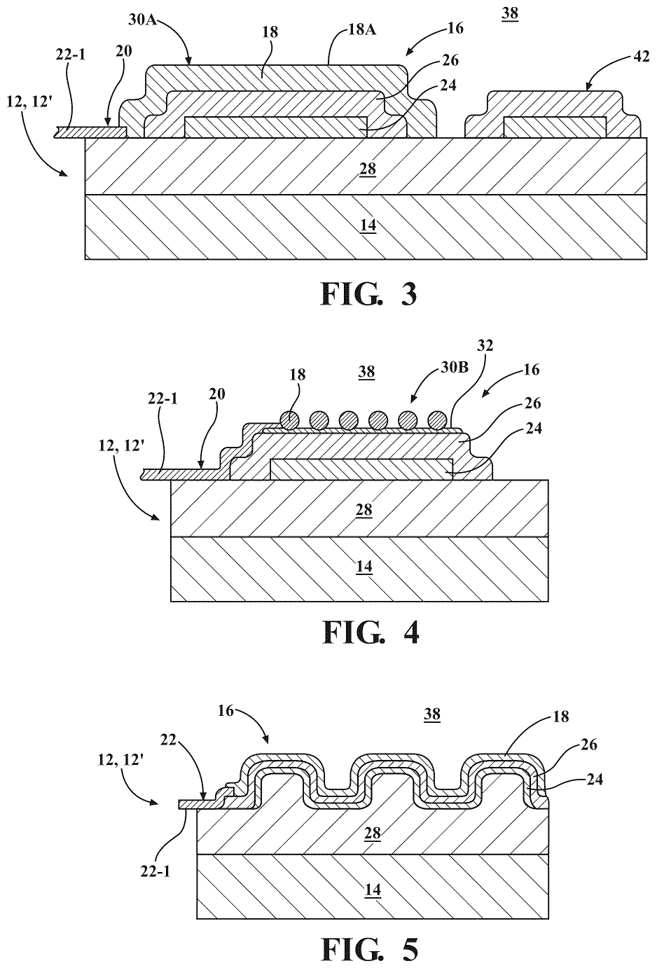

Referring to FIGS. 1A and 1B, a gas sensing system 10 having at least one gas sensing microchip device 12 is shown. Each microchip device 12 includes a substrate layer 14, which may be constructed from silicon. The substrate layer 14 may be electrically grounded to the structure of system 10. As shown in FIGS. 2 and 3, the microchip device 12 also includes a gas capacitor (GASCAP) 16. The GASCAP 16 includes a first electrically conductive plate 18 configured, i.e., constructed and arranged, to operate as a catalyst when interacting with one or more gases. The first plate 18 may is configured to adsorb specific gas(es) via an exposed surface 18A. Such adsorption of gas(es) by the exposed gas sensing surface 18A is intended to facilitate detection or sensing of the subject gas(es) by generating electrical energy on the first electrically conductive plate 18. The first electrically conductive plate 18 may be constructed from metal or another (nonmetallic) material defined by a work-function property reversibly responsive to gas(es) that are sought to be detected. The material of the first electrically conductive plate 18 may be Palladium, Platinum, Gold, Silver, Ruthenium, Silver/Gold, or another active metal.

The GASCAP 16 also includes a gas-insensitive, electrically conductive element 20 electrically connected to, e.g., in physical contact with, the first electrically conductive plate 18. The element 20 includes a first electrical terminal 22-1. The element 20 is configured to be either electrically grounded or biased at a fixed voltage, such that the element 20 does not exhibit floating potential. However, the fixed voltage should be sufficiently low so that it does not cause dielectric material breakdown. The element 20 may be constructed from metal and be minimally overlapped by the first plate 18 to maintain continuous contact therebetween. The GASCAP 16 additionally includes a gas-insensitive or inert second electrically conductive plate 24. The second plate 24 includes a second electrical terminal 22-2 (shown in FIG. 7) and may be constructed from metal. Each of the electrically conductive element 20 and the gas-insensitive second electrically conductive plate 24 may be constructed from same metal, such as Tungsten, Titanium/Nickel, or Aluminum/Copper.

Each of the electrically conductive element 20 and second electrically conductive plate 24 may, for example, have a thickness in a 50 nm-100 nm range. Furthermore, the first electrically conductive plate 18 and the electrically conductive element 20 may be defined by any geometric shape, i.e., square, rectangular, circular, etc. Depending on the gas to be sensed, the first electrically conductive plate 18 may be a uniform layer of a single metal or a mixture/alloy of multiple metals. Depending on the type of metal(s) used for the first electrically conductive plate 18, specific gases can reversibly change the work-function property of the subject metal(s), which is an inherent material property and a key aspect governing the underlying sensing principle.

As such, the GASCAP 16 includes two opposing capacitor plates—the first electrically conductive plate 18 and the second electrically conductive plate 24. The first electrically conductive plate 18 and the electrically conductive element 20 together define a first capacitor plate area CP_{A1} and the second electrically conductive plate 24 define a second capacitor plate area CP_{A2}. The relationship between CP_{A1} and CP_{A2} is understood to impact sensing performance of the microchip device 12. Structuring the GASCAP 16 with the first capacitor plate area CP_{A1} greater than the second capacitor plate area CP_{A2} is expected to enhance sensitivity of the GASCAP to the selected gas(es). The GASCAP 16 further includes a comparatively high permittivity dielectric, i.e., an insulator with high polarizability, layer 26 arranged between the first electrically conductive plate 18 and the second electrically conductive plate 24.

In general, permittivity (ε) is the ability of a material to store electrical energy in an electric field. In electromagnetism, permittivity is a measure of the electric polarizability of a dielectric material. A material with high permittivity polarizes more in response to an applied electric field than a material with low permittivity, thereby storing more energy in the material. In electrostatics, permittivity plays an important role in determining the capacitance of a capacitor. Permittivity of the dielectric layer 26 may be in a range of 8-60 relative to permittivity of free space (vacuum) or in absolute values in a range of 7.0832e⁻¹¹ F/m-5.3124e⁻¹⁰ F/m. The high permittivity dielectric layer 26 may be constructed from materials such as Alumina (Al₂O₃), Hafnia (HfO₂), Zirconia (ZrO₂), Hafnium Zirconate (HfZrO₂ or HfZrO₄), and Hafnium silicate (HfSiO₂).

The dielectric layer 26 is configured, i.e., arranged and employed, to induce a capacitive charge distribution on the gas-insensitive second electrically conductive plate 24 and an electrical bias or potential between the first and second electrically conductive plates 18, 24 in response to the gas(es) being detected by the first plate 18. The induced charge distribution and electrical bias in turn generates an electrical current flow between the first and second electrical terminals 22-1, 22-2, corresponding to an amount of the gas(es) being detected. The electrical current flow generated due to the induced electrical bias is transient, occurring while the gas(es) is being adsorbed. As shown in FIGS. 2 and 3, the first plate 18 may overlap the electrically conductive element 20, such that element 20 is in direct contact with the comparatively high permittivity dielectric layer 26. Furthermore, the area of overlap between the first plate 18 and the electrically conductive element 20 is minimized to ensure maximum coverage of the dielectric layer 26 by the first plate. For example, the geometric area of the first plate 18 may be at least 10 times greater than the area of overlap between the first plate and the electrically conductive element 20.

With continued reference to FIGS. 2 and 3, the microchip device 12 also includes a comparatively low permittivity dielectric, i.e., an insulator with low polarizability, layer 28. Permittivity of the dielectric layer 28 may be in a range of 2-4 relative to permittivity of vacuum or in absolute values in a range of 1.77e⁻¹¹ F/m-3.54e⁻¹¹ F/m. In other words, to minimize parasitic capacitances, comparatively, the permittivity of the dielectric layer 26 is at least two times greater than the permittivity of the dielectric layer 28. For example, the comparatively high permittivity dielectric layer 26 and the comparatively low permittivity dielectric layer 28 may each be defined by a leakage current density of ≤1 pA/cm².

The comparatively low permittivity dielectric layer 28 may be constructed from a low thermal conductivity material and is generally characterized by sufficient thickness to prevent formation of an electric field causing electrical interference affecting the second electrically conductive plate 24. The dielectric layer 28 is arranged between the substrate layer 14 and the second electrically conductive plate 24 and configured to isolate the GASCAP 16 thermally and electrically from the substrate layer. Also, as shown (in FIG. 3), the first electrically conductive plate 18 may extend to the dielectric layer 28 and thereby encapsulate, i.e., cover entirely, the dielectric layer 26 without electrically shorting or connecting to second electrically conductive plate 24.

The first electrically conductive plate 18 may have an electrically continuous, e.g., flat sheet or unbroken mesh, structure 30A (shown in FIGS. 2 and 3). Alternatively, the first electrically conductive plate 18 may have an electrically discontinuous, e.g., nanoparticle or nanowire structure 30B (shown in FIG. 4). For example, the diameters of the subject nanoparticles or nanowires may be in a range of 5-10 nm. In GASCAP 16 having the first electrically conductive plate 18 with the electrically discontinuous structure 30B, the GASCAP may additionally include a gas-insensitive electrically conductive charge transfer layer (CTL) 32 (shown in FIG. 4). The CTL 32 may be arranged in direct contact with the electrically conductive element 20, between the electrically discontinuous first electrically conductive plate 30B and the dielectric layer 26. Employing the CTL 32 with the discontinuous first electrically conductive plate 18 electrically connects the discontinuous first plate to ground or set to a fixed potential.

The CTL 32 may have a limited thickness ≤10 nm to ensure the CTL doesn't block out of the electric field response to changes in the work-function of the first electrically conductive plate 18 during gas detection. The CTL 32 may be constructed from a gas insensitive metal, such as Ti, Ni, W, Cu, etc. Alternatively, the CTL 32 may be constructed from a metal oxide which has nonexistent or minimal gas sensitivity at low temperatures (<100° C.), such as Indium Gallium Zinc Oxide (IGZO), Zinc Oxide (ZnO), Titanium Oxide (TiO₂), Tin (IV) oxide (SnO₂), Indium Oxide (InO₂), etc.). As shown in FIG. 5, the gas sensing surface 18A of the first electrically conductive plate 18 may be non-planar, e.g., have a corrugated form or pattern. Furthermore, the first electrically conductive plate 18 is characterized by a uniform material thickness. In such an embodiment, each of the second electrically conductive plate 24 and the comparatively high permittivity dielectric layer 26, as well as the adjoining surface of the comparatively low permittivity dielectric layer 28, may also be non-planar. Such a construction of the respective GASCAP 16 elements is configured to enhance total capacitance of the GASCAP without increasing its overall volume and packaging space occupied by the microchip device 10.

With reference to FIG. 7, the fundamental operating principle of GASCAP 16 is storage of electrical energy in response to interaction with gas(es). GASCAP 16 is a DC voltage driven device that stores electrical energy by accumulating electric charges at a given potential on opposite GASCAP surfaces that are separated by an insulating layer. Interaction of a particular gas with the first electrically conductive plate 18 induces charges thereon. In response, electric dipoles inside the comparatively high permittivity dielectric layer 26 re-orient and align themselves, such that equal and opposite polarity charges are induced on the gas-insensitive second electrically conductive plate 24.

In an ideal scenario, with both electrically conductive plates 18, 24 connected to some potential or grounded, whenever there is a gas present and interacting with the gas sensitive first electrically conductive plate 18, a transient current will flow between the potential/ground source terminal and the two electrically conductive plates. In other words, ideally, the subject current does not flow through the comparatively high permittivity dielectric layer 26, as the subject dielectric layer is an electrical insulator. Moreover, the subject current, being transient, will flow for a brief duration of time, specifically until the charge on the first electrically conductive plate 18 is balanced by equal and opposite charges on the second electrically conductive plate 24.

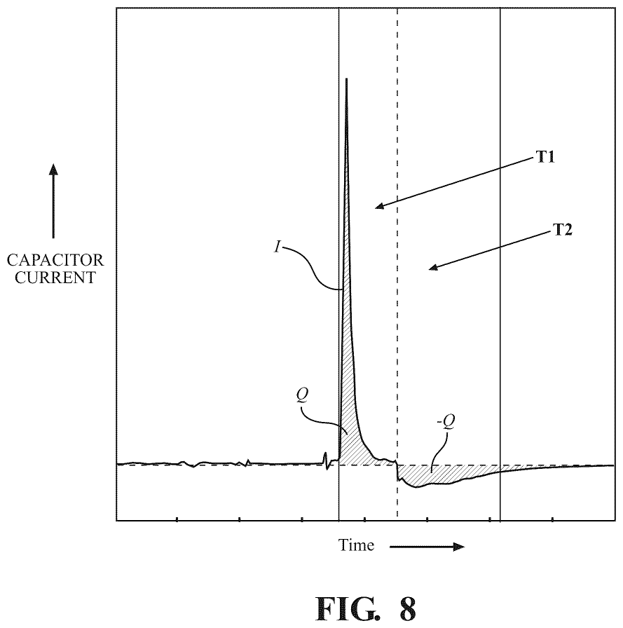

FIG. 8 depicts transient current flow (I) across the GASCAP 16 within region T1 as a result of the sensed gas being adsorbed onto the first electrically conductive plate when the first electrically conductive plate 18 is initially exposed to the gas and then trailing off to zero over time. The polarity of transient current flow (I) may be positive (or negative) depending on the nature of the specific interaction of the gas and the first electrically conductive plate 18. Following cessation of the first electrically conductive plate's exposure to the sensed gas in region T2, transient current flow (I) may change polarity for a brief period of time as a result of the sensed gas being desorbed from the first electrically conductive plate 18 and then trail off to zero. Both the gas adsorption and desorption may be correlated with a charge (Q) generated on the first electrically conductive plate 18 and the second electrically conductive plate 24. The charge (Q) has equal magnitude but opposite polarity in regions T1 and T2 and may be determined by integration of the shaded area under the current flow (I) curve.

The DC voltage may be duty cycled, i.e., the voltage may be applied for short bursts, measured (at V_{M1} and V_{M2}, shown in FIG. 7) and then turned off-which may reduce long term drift of the GASCAP 16. The total voltage developed across the GASCAP 16 is V_{GASCAP}=|V_{M2}−V_{M1}|. Whenever a target gas is present in the surrounding environment 38, the gas will be molecularly adsorbed through the exposed gas sensing surface 18A for interaction (via dissociation or physisorption) with the first electrically conductive plate 18. Such an interaction of the gas with the material of the first electrically conductive plate 18 will result in a modification or change of the first electrically conductive plate material's catalytic work-function (ΔØ_{CAT}=Ø′_{CAT}−Ø_{CAT}), which is an inherent property of the subject material. The work-function change ΔØ_{CAT} is associated with surface and/or bulk material effects and charge redistribution in the first electrically conductive plate 18.

The work-function change ΔØ_{CAT} causes a pseudo/intrinsic potential difference to develop across the dielectric layer 26 and relative to the second electrically conductive plate 24. An electric field is formed due to the potential difference and results in an equal and opposite transient current (I) to flow between the source V_{M2} connected to the top plate and the source V_{M1} connected to the bottom plate. The subject phenomenon is analogous to the charging/discharging transient displacement current in conventional electronic capacitors. However, in the case of the GASCAP 16, gas molecule adsorption/desorption on the first electrically conductive plate 18 “charges” and “discharges” the GASCAP.

Depending on the type of gas and the first electrically conductive plate 18 material, the subject work-function change ΔØ_{CAT} may be a positive (higher) value or a negative (lower) value. The adsorption transient current (I′) will continue to flow until the work-function of the first electrically conductive plate 18 (and by extension the electric field across the dielectric layer 26) stops changing, at which point, the adsorption current (I′) will ideally return to zero (in practice, some very low non-zero current may remain). The first electrically conductive plate's work-function will stop changing when the amount of adsorbed gas reaches equilibrium with the amount of gas present in the surrounding environment. The subject characteristic of the adsorption transient current (I) returning to a zero value, may endow the GASCAP 16 with ideally zero or negligible drift, simplifying GASCAP calibration and resulting in a precise, stable, and reliable gas sensing device. Compared to GASCAP 16, existing gas sensing solutions require complex compensation using hardware and software controls to minimize drift.

The exact mode of interaction between a gas and the first electrically conductive plate 18 is dependent on the type of gas and the first electrically conductive plate material. For example, in the case of a Palladium (Pd) first electrically conductive plate 18 and a hydrogen gas, adsorbed and disassociated atomic hydrogen will diffuse through the Pd first electrically conductive plate to affect the dipole between the Pd and the adjacent dielectric layer 26 in addition to causing lattice expansion of the Pd. In the case of a Gold (Au) first electrically conductive plate 18 and a sulfide gas (for example hydrogen sulfide or tert-butyl mercaptan), the sulfide gas may only adsorb on the exposed Au gas sensing surface 18A and not diffuse through or cause lattice expansion in the Au first electrically conductive plate. However, the net effect from the target gas and specific gas sensing surface 18A interaction is a work-function change inducing a sufficiently strong change in the local electric field across the dielectric layer 26 and relative to the second electrically conductive plate 24.

Integrating the capacitor current over time provides the total charge (Q1) generated by the gas-induced work-function change ΔØ_{CAT} in the first electrically conductive plate 18. When the target gas molecules are no longer present in the atmosphere, adsorbed target gas molecules will desorb at some rate, causing the first electrically conductive plate's work function to revert to its state prior to the introduction of the target gas. Just like in the gas adsorption case, desorption is accompanied with an electric field change leading to equal and opposite transient current (I″) flowing between the source V_{M2} connected to the first electrically conductive plate 18 and the source V_{M1} connected to the second electrically conductive plate 24.

The polarity of the currents when a detected gas is present (adsorbing on the first electrically conductive plate 18) versus when such a gas is no longer present (desorbing from the first electrically conductive plate), will be opposite. Analogously to the above description, the desorption transient current (I″) will flow as long as the first electrically conductive plate's work-function is changing and reverting to its initial/original state, prior to the presence of the target gas. Once the work-function stops changing, the desorption transient current (I″) will ideally return to zero. Integrating the desorption transient current (I″) over time results in the total charge (Q2) removed from the first electrically conductive plate 18 when the gas molecules are no longer present (i.e., leaving/desorbing the first electrically conductive plate). Overall, the total charge |Q1| will be equal to total charge |Q2|, and the polarity of the two transient currents is opposite, i.e., if (I′) was positive, (I″) will be negative, and vice-versa.

With resumed reference to FIGS. 1A and 1B, the gas sensing system 10 also includes an electronic control unit (ECU) 34 in operative communication with the microchip device 12. The ECU 34 is configured, i.e., constructed and programmed, to ascertain the amount of the gas(es) being detected by the first electrically conductive plate 18 and corresponding to the transient electrical current flow generated between the first and second electrical terminals 22-1, 22-2. The microchip device 12 may be either integrally formed with or physically wired to the ECU 34. ECU 34 is intended to include a processor and tangible, non-transitory memory, with instructions for managing operation of the microchip device 12 programmed therein. The ECU's memory may be an appropriate recordable medium that participates in providing computer-readable data or process instructions. Such a recordable medium may take many forms, including but not limited to non-volatile media and volatile media.

Non-volatile media for the ECU 34 may include persistent memory accessible via appropriate memory instructions. Volatile media may include, for example, dynamic random-access memory (DRAM), which may constitute a main memory. The instructions programmed into the ECU 34 may be transmitted by an appropriate transmission medium, such as wiring and fiber optics, or via a wireless connection. The ECU 34 may be equipped with other required computer hardware, such as a high-speed clock, requisite Analog-to-Digital (A/D) and/or Digital-to-Analog (D/A) circuitry, input/output circuitry and devices (I/O), as well as appropriate signal conditioning and/or buffer circuitry. Algorithm(s), indicated generally via numeral 36, required by the ECU 34 or accessible thereby may be programmed into the ECU, stored within its memory, and automatically executed to facilitate operation of the gas sensing system 10.

The ECU 34 is typically configured to receive a signal generated by the microchip device 12 indicative of a detected amount of corresponding gas(es) in a confined environment 38. For example, the signal may be respective of the amount of gas(es) detected within an enclosure of a rechargeable energy storage system (RESS). The algorithm(s) 36 may also include instructions to compare the amount of gas(es) detected by the microchip device 12 to a predetermined threshold amount of the subject gas(es) programmed into the ECU 34. The algorithm(s) 36 may include further instructions to trigger a signal indicative of a critical condition being present in the confined environment 38. For example, such a critical condition may be an impending failure of a monitored RESS, as indicated by the detected amount of one or more of the subject gases exceeding the respective predetermined threshold amount(s).

The gas sensing system 10 may additionally include a variable capacitance device 40, such as a MOSFET or a MOSCAP. As generally understood, a MOSFET is a metal-oxide-semiconductor field-effect transistor (FET). A MOSFET has an insulated gate, the voltage of which determines the conductivity of the device. This ability to change conductivity with the amount of applied voltage may be used for amplifying or switching electronic signals. A MOSFET includes a source, a drain, a gate, and a semiconductor substrate forming a channel that connects the source and the drain. The gate is separated from the channel by a thin layer of oxide (usually silicon dioxide). The MOSFET structure allows for the control of current flow between the source and drain by applying a voltage to the gate. Operation of a MOSFET is based on an electric field created by the gate voltage, which modulates the conductivity of the channel between the source and drain.

As generally understood, a MOSCAP is a metal-oxide-semiconductor (MOS) capacitor. A MOSCAP typically includes a metal gate, an insulating oxide layer, and a semiconductor substrate. Unlike a MOSFET, a MOSCAP has no source or drain. A MOSCAP is primarily focused on capacitive behavior, rather than on current amplification or switching. A MOSCAP functions primarily as a capacitor. The capacitance of a MOSCAP is determined by the area of the gate and the thickness of the oxide layer. A MOSCAP is typically used to study electrical properties of semiconductor materials and for applications like charge storage, voltage tuning, and memory devices. While both MOSFETs and MOSCAPs utilize a similar metal-oxide-semiconductor structure, a MOSFET is designed for switching and amplification of electrical signals, whereas a MOSCAP focuses on capacitive storage and analysis of semiconductor characteristics.

In the subject embodiment, the variable capacitance device 40 may be operatively connected to the second electrical terminal 22-2 (shown in FIG. 7), such that the gas insensitive second electrically conductive plate 24 is electrically floating, i.e., is not connected to any specific potential (voltage). Thus connected, the variable capacitance device 40 may be configured to respond with a change in threshold voltage indicative of the capacitive charge distribution on the second plate 24. For example, the second electrical terminal 22-2 may be connected to the gate terminal of the MOSFET, such that the MOSFET is connected in series with the GASCAP capacitance.

In another example, the second electrical terminal 22-2 may be connected to the gate terminal of the MOSCAP, such that the MOSCAP is connected in series with the GASCAP capacitance. As shown in FIG. 2, the variable capacitance device 40 may be arranged on, e.g., mounted to, the substrate layer 14 and below the relatively low permittivity dielectric layer 28, such that, for example for a MOSFET, the electrical wiring from the source and gate and drain terminals are routed through the low permittivity dielectric layer 28. Such construction would generate an integrated, monolithic system-on-chip (SoC) 12A. Alternatively, as shown in FIGS. 1A and 7, the variable capacitance device 40 may be arranged remotely from the microchip device 12, while being in electrical connection therewith.

With resumed reference to FIG. 3, the gas sensing system 10 may additionally include a reference capacitor 42 arranged proximately to the GASCAP 16 and exposed to the same confined environment 38. The reference capacitor 42 may have a structure similar to (with comparable elements being substantially alike) the GASCAP 16, but with the first electrically conductive plate 18 of the reference capacitor being gas insensitive. The first electrically conductive plate 18 of the reference capacitor 42 may be constructed from the same material as the gas insensitive second electrically conductive plate 24. Alternately the first electrically conductive plate 18 of the reference capacitor 42 may be constructed from the same material as the gas sensitive first electrically conductive plate 18 of the GASCAP 16 but covered entirely by another material similar to the gas insensitive second electrically conductive plate 24.

The reference capacitor 42 may be in operative communication with another variable capacitance device, which is similar to device 40 but is not electrically connected to the GASCAP 16. The reference capacitor 42 may be specifically configured to compensate for electronic drift of the GASCAP 16 during detection of the gas(es). As shown in FIG. 2, the gas sensing system 10 may also include a microheater 44, e.g., resistor-based, arranged next to GASCAP 16 or between the GASCAP and the reference capacitor 42. The microheater 44 may be configured to generate thermal energy and thereby increase the temperature of the GASCAP 16, as well as the reference capacitor 42. The gas sensing system 10 may additionally include a temperature sensor 46 (shown in FIG. 2) arranged proximately to the GASCAP 16 and the reference capacitor 42 and configured to detect temperature of the GASCAP.

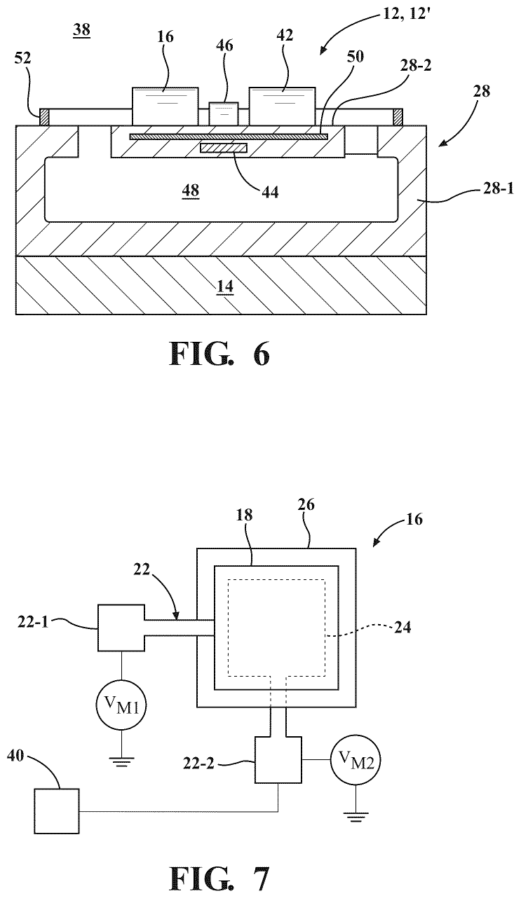

As shown in FIG. 6, the comparatively low permittivity dielectric layer 28 may include a comparatively low permittivity dielectric layer base 28-1 and a comparatively low permittivity dielectric layer island or membrane 28-2 mechanically suspended thereon. The dielectric layer island 28-2 may be arranged on top of the SoC 12A and separated therefrom by an airgap or a recessed cavity 48. The dielectric layer island 28-2 may be constructed from the comparatively low permittivity dielectric material characterized by low thermal mass and conductivity (≤10 W/(m·K). The dielectric layer island 28-2 may be configured to support each of the GASCAP 16, the reference capacitor 42, the microheater 44, and the temperature sensor 48. Meanwhile, the variable capacitance device 40 may be arranged below the dielectric base layer 28-1 and incorporated into the substrate layer 14, such that, for example, if the variable capacitance device 40 is a MOSFET, the electrical wiring from the source and gate and drain terminals are routed through the low permittivity dielectric base layer.

The recessed cavity 48 may be configured to minimize thermal losses of the microheater 44 and overall power consumption of the microchip device 12. The suspended dielectric layer island 28-2 construction may therefore enhance operating efficiency of the microchip device 12 by having thermal energy retained in the island, without the power required to heat the GASCAP 16 to operating temperature dissipated to surrounding areas. The gas sensing microchip device 12 may also include a heat spreading structure 50, e.g., constructed from metal, embedded in the dielectric layer island 28-2. The heat spreading structure 50 may be configured to generate a uniform temperature profile or distribution across the dielectric layer island 28-2, thus enhancing efficiency of the GASCAP 16. The gas sensing microchip device 12 may additionally include a guard ring 52 arranged on the dielectric layer base 28-1. The guard ring 52 may surround each of the GASCAP 16 and the reference capacitor 42 and be configured to minimize stray charge build-up or electromagnetic interference (EMI) on each of the GASCAP and the reference capacitor. The guard ring 52 may be constructed from a gas insensitive metal, such as the material of the second electrically conductive plate 24 and may be electrically floating, grounded, or biased.

Algorithm(s) 36 programmed into the ECU 34 may include instructions to perform regeneration of the GASCAP 16 for removing contaminants (dust particles, charged particles, moisture, water vapor, etc.) therefrom that negatively affect detection sensitivity of the gas sensing microchip device 12. Regeneration of the GASCAP 16 may be performed by monitoring operating or sensing temperature of the GASCAP via the temperature sensor 46 and increasing the temperature of the GASCAP via the microheater 44. The regeneration temperature provided by the microheater 44 may be in a range of 100 to 300+ degrees Celsius. The ECU 34 may be configured to determine, i.e., decode or interpret, the operating temperature detected by the temperature sensor 46 using a relationship between electrical resistance of the temperature sensor and the temperature of the GASCAP 16 programmed into the ECU.

The gas sensing system 10 may include a number of microchip devices 12 arranged as a group of individual microchips connected to the ECU 34 (shown in in FIG. 1A) or be incorporated into a unitary sensor array microchip 12′ (shown in in FIG. 1B). Either of such embodiments of the gas sensing system 10 may be configured to detect multiple distinct gases present in the surrounding environment 38. In such structures, each individual GASCAP 16 may be configured to detect a selected combination of gases or one of such distinct gases and communicate the subject data to the ECU 34.

Overall, the gas sensing system 10 employs one or more microchip devices 12 with GASCAP(s) 16 to detect presence of gas(es) in a chosen environment. The gas sensing system 10 includes an ECU to assess amount(s) of gas(es) detected by the microchip device(s) 12. Each microchip device 12 may include microheater(s) and temperature sensor(s) to maintain operating temperature and perform regeneration of the GASCAP(s). The gas sensing system 10 may also include reference capacitor(s) 42 configured to compensate for electronic drift of the GASCAP during detection of the gas(es). The gas sensing system 10 may further include at least one variable capacitance device (one for each of GASCAP 16 and reference capacitor 42) configured to respond to capacitive charge distribution in the GASCAP or in the reference capacitor with a change in threshold voltage of the corresponding variable capacitance device.

The detailed description and the drawings or figures are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings, or the characteristics of various embodiments mentioned in the present description are not necessarily to be understood as embodiments independent of each other. Rather, it is possible that each of the characteristics described in one of the examples of an embodiment may be combined with one or a plurality of other desired characteristics from other embodiments, resulting in other embodiments not described in words or by reference to the drawings. Accordingly, such other embodiments fall within the framework and the scope of the appended claims.

What is claimed is:

1. A gas sensing microchip device, comprising:
a substrate layer;
a gas capacitor (GASCAP) including:
a first electrically conductive plate configured to interact with and detect at least one gas via an exposed gas sensing surface;
a gas-insensitive, electrically conductive element electrically connected to the first electrically conductive plate and having a first electrical terminal;
a gas-insensitive second electrically conductive plate having a second electrical terminal; and
a comparatively high permittivity dielectric layer arranged between the first and second electrically conductive plates and configured to induce a capacitive charge distribution on the gas-insensitive second electrically conductive plate and an electrical bias between the first and second electrically conductive plates in response to the at least one gas being detected by the first electrically conductive plate, thereby generating a transient electrical current flow between the first and second electrical terminals corresponding to an amount of the at least one gas being detected; and
a comparatively low permittivity dielectric layer arranged between the substrate layer and the second electrically conductive plate and configured to isolate the GASCAP thermally and electrically from the substrate layer.

2. The gas sensing microchip device of claim 1, wherein the first electrically conductive plate is constructed from a metal defined by a work-function property reversibly responsive to the at least one gas.

3. The gas sensing microchip device of claim 1, wherein the first electrically conductive plate extends to the comparatively low permittivity dielectric layer and thereby encapsulates the comparatively high permittivity dielectric layer.

4. The gas sensing microchip device of claim 1, wherein the first electrically conductive plate and the electrically conductive element together define a first capacitor plate area and the gas-insensitive second electrically conductive plate defines a second capacitor plate area, and wherein the first capacitor plate area is greater than the second capacitor plate area, thereby enhancing sensitivity of the GASCAP to the at least one gas.

5. The gas sensing microchip device of claim 1, wherein the first electrically conductive plate has an electrically discontinuous structure.

6. The gas sensing microchip device of claim 5, further comprising a gas-insensitive electrically conductive charge transfer layer arranged in direct contact with the electrically conductive element, between the electrically discontinuous first electrically conductive plate and the dielectric layer.

7. The gas sensing microchip device of claim 1, wherein the first electrically conductive plate has an electrically continuous structure.

8. The gas sensing microchip device of claim 1, wherein the gas sensing surface of the first electrically conductive plate is non-planar and thereby configured to enhance total capacitance of the GASCAP without increasing overall volume and packaging space occupied by the microchip device.

9. The gas sensing microchip device of claim 1, further comprising a variable capacitance device operatively connected to the second electrical terminal, wherein the variable capacitance device is configured to respond with a change in threshold voltage indicative of the capacitive charge distribution on the gas-insensitive second electrically conductive plate.

10. The gas sensing microchip device of claim 9, further comprising a reference capacitor arranged proximately to the GASCAP, in operative communication with the variable capacitance device, and configured to compensate for electronic drift of the GASCAP during detection of the at least one gas.

11. The gas sensing microchip device of claim 10, further comprising:
a microheater configured to generate thermal energy and thereby increase temperature of the GASCAP and the reference capacitor; and
a temperature sensor configured to detect temperature of the GASCAP.

12. The gas sensing microchip device of claim 11, wherein the comparatively low permittivity dielectric layer includes a comparatively low permittivity dielectric layer base and a comparatively low permittivity dielectric layer island mechanically suspended thereon and separated therefrom by an airgap, wherein the comparatively low permittivity dielectric layer island is configured to support the GASCAP, the reference capacitor, the microheater, and the temperature sensor.

13. A gas sensing system, comprising:
a gas sensing microchip device including:
a substrate layer;
a gas capacitor (GASCAP) including:
a first electrically conductive plate configured to interact with and detect at least one gas via an exposed gas sensing surface;
a gas-insensitive, electrically conductive element electrically connected to the first electrically conductive plate and having a first electrical terminal;
a gas-insensitive second electrically conductive plate having a second electrical terminal; and
a comparatively high permittivity dielectric layer arranged between the first and second electrically conductive plates and configured to induce a capacitive charge distribution on the gas-insensitive second electrically conductive plate and an electrical bias between the first and second electrically conductive plates in response to the at least one gas being detected by the first electrically conductive plate, thereby generating a transient electrical current flow between the first and second electrical terminals corresponding to an amount of the at least one gas being detected; and
a comparatively low permittivity dielectric layer arranged between the substrate layer and the second electrically conductive plate and configured to isolate the GASCAP thermally and electrically from the substrate layer; and
an electronic control unit (ECU) in operative communication with the gas sensing microchip device and configured to ascertain the amount of the at least one gas being detected by the first electrically conductive plate and corresponding to the transient electrical current flow generated between the first and second electrical terminals.

14. The gas sensing system of claim 13, wherein the first electrically conductive plate is constructed from a metal defined by a work-function property reversibly responsive to the at least one gas.

15. The gas sensing system of claim 13, wherein the first electrically conductive plate extends to the comparatively low permittivity dielectric layer and thereby encapsulates the comparatively high permittivity dielectric layer.

16. The gas sensing system of claim 13, wherein the first electrically conductive plate and the electrically conductive element together define a first capacitor plate area and the gas-insensitive second electrically conductive plate defines a second capacitor plate area, and wherein the first capacitor plate area is greater than the second capacitor plate area, thereby enhancing sensitivity of the GASCAP to the at least one gas.

17. The gas sensing system of claim 13, wherein the first electrically conductive plate has an electrically discontinuous structure.

18. The gas sensing system of claim 17, further comprising a gas-insensitive electrically conductive charge transfer layer arranged in direct contact with the electrically conductive element, between the electrically discontinuous first electrically conductive plate and the dielectric layer.

19. The gas sensing system of claim 13, wherein the first electrically conductive plate has an electrically continuous structure.

20. The gas sensing system of claim 13, wherein the gas sensing surface of the first electrically conductive plate is non-planar and thereby configured to enhance total capacitance of the GASCAP without increasing overall volume and packaging space occupied by the microchip device.

21. The gas sensing system of claim 13, further comprising a variable capacitance device operatively connected to the second electrical terminal, wherein the variable capacitance device is configured to respond with a change in threshold voltage indicative of the capacitive charge distribution on the gas-insensitive second electrically conductive plate.

22. The gas sensing system of claim 21, wherein the variable capacitance device is arranged remotely from the gas sensing microchip device.

23. The gas sensing system of claim 21, further comprising a reference capacitor arranged proximately to the GASCAP, in operative communication with the variable capacitance device, and configured to compensate for electronic drift of the GASCAP during detection of the at least one gas.

24. The gas sensing system of claim 23, further comprising:
a microheater configured to generate thermal energy and thereby increase temperature of the GASCAP and the reference capacitor; and
a temperature sensor configured to detect temperature of the GASCAP.

25. The gas sensing system of claim 24, wherein the dielectric layer includes a dielectric layer base and a dielectric layer island mechanically suspended thereon and separated therefrom by an airgap, wherein the dielectric layer island is configured to support the GASCAP, the reference capacitor, the microheater, and the temperature sensor.
